(19) United States
(12) Patent Application Publication
(10) Pub. No.: US 2025/0127512 A1
(43) Pub. Date: Apr. 24, 2025

(54) SYSTEMS AND METHODS FOR STIMULATION, NERVE REPAIR AND/OR DRUG DELIVERY

(71) Applicants: The Brigham and Women's Hospital, Inc., Boston, MA (US); Massachusetts General Hospital, Boston, MA (US); Massachusetts Institute of Technology, Cambridge, MA (US)

(72) Inventors: Shriya Srinivasan, Cambridge, MA (US); Paramesh Karandikar, Boston, MA (US); Lisa Gfrerer, Boston, MA (US); Giovanni Traverso, Cambridge, MA (US); Robert Langer, Boston, MA (US); Avik Som, Boston, MA (US)

(21) Appl. No.: 18/688,218

(22) PCT Filed: Sep. 2, 2022
(86) PCT No.: PCT/US2022/042540

§ 371 Date: Feb. 29, 2024

Related U.S. Application Data
(60) Provisional application No. 63/240,061, filed on Sep. 2, 2021.

(51) Int. Cl. A61B 17/11; A61B 17/00; A61L 27/26; A61L 27/52; A61L 27/54; A61L 27/58; A61N 1/05; A61N 1/36

(52) U.S. Cl. CPC A61B 17/1128; A61L 27/26; A61L 27/52; A61L 27/54; A61L 27/58; A61N 1/36103; A61B 2017/00004; A61B 2017/00893; A61L 2430/32; A61N 1/0551

(57) ABSTRACT

Systems and methods for nerve repair can include a polymer configured to form an elongated conduit to receive an in vivo nerve therein and provide a mechanical stabilization to the in vivo nerve. The polymer can be configured to degrade upon being subjected to a dissolution solution to remove the mechanical stabilization from the in vivo nerve.

### CROSS-REFERENCES TO RELATED APPLICATIONS

The present application is based on and claims priority from U.S. Patent Application No. 63/240,061, filed on Sep. 2, 2021, the entire disclosure of which is incorporated herein by reference.

### STATEMENT REGARDING FEDERALLY SPONSORED RESEARCH

Not Applicable.

### BACKGROUND

With over twenty million peripheral nerve injuries yearly, the healthcare burden of such injuries can project upwards of $150 billion USD. The primary consequences to the patient are debilitating losses of motor and sensory function, which result in great loss of mobility, capabilities, independence and ultimately hinder the quality of life for patients. Based on the level of trauma, the consequences range from pain and tingling to severe muscle atrophy and even paralysis. In some analyses of median and ulnar nerve injuries, it was observed that only approximately 51.6% of cases achieve satisfactory motor recovery, and approximately 42.6% of cases experience satisfactory sensory recovery with current and conventional surgical and therapeutic methods.

Electrical stimulation (ES) has been known to accelerate axonogenesis by modulating plasticity, elevating neuronal cyclic adenosine monophosphate and upregulating neurotrophic factors in neurons and Schwann cells. ES can improve muscle mass and prevent atrophy by preventing apoptosis of denervated muscle fibers. In various clinical trials, ES has demonstrated significant improvements in sensory and motor function whereas surgery alone had marginal effects. For example, to prevent muscle atrophy, transcutaneous electrical stimulation can be used (TENS systems). However, patient compliance and pain at the surface of the skin pose challenges. As such, TENS systems, and other ES systems designed for transcutaneous electrical stimulation, are ineffective for clinical applications that require ES at any appreciable distance from the skin.

In some limited circumstances, electrodes have been implanted into or proximate an internal site. However, with conventional techniques and technology, scarring and adhesion to the implant is extremely common and unwanted.

Thus, there is a need for systems and methods that address these and other drawbacks of expediting therapeutic processes at internal locations in the body, particularly, for delicate clinical applications, such as nerve stimulation and nerve repair.

### SUMMARY

In some aspects, the present disclosure provides a nerve repair system. The nerve repair system can include a scaffold that can be positioned around a nerve repair site. The scaffold can be dissolvable, for example, such as when exposed to a dissolution solution. In one non-limiting example, a set of probe(s) can be secured relative to the nerve repair site via the scaffold. The probe or probes can be configured to deliver stimulus. In one non-limiting example, the stimulus could be electrical stimulus or optical stimulus. The scaffold can facilitate atraumatic removal of the probes adjacent to the nerve and the nerve repair site and the scaffold can be configured to dissolve after a nerve repair procedure has been completed. In another non-limiting example, the scaffold can be accompanied by a drug to the nerve repair. In yet another non-limiting example, the scaffold may be designed to engage the nerve to facilitate alignment and positioning, or other mechanical stabilization for repair, with or without stimulation or drug delivery.

Some aspects of the disclosure provide a system for nerve repair. The system can include a polymer that is configured to form an elongated conduit that forms a lumen configured to receive an in vivo nerve therein and present a mechanical stabilization to the in vivo nerve. The polymer can be configured to degrade upon being subjected to a dissolution solution to remove the mechanical stabilization from the in vivo nerve.

Some aspects of the disclosure provide a kit. The kit can include a polymer configured to form an elongated conduit that forms a lumen. The lumen can be configured to receive an in vivo nerve therein and present a mechanical stabilization to the in vivo nerve. A dissolution solution can be configured to dissolve the polymer and remove the mechanical stabilization from the in vivo nerve upon being delivered to the polymer.

Other aspects of the disclosure will be provided as follows, each of which is non-limiting to any particular invention.

### BRIEF DESCRIPTION OF THE DRAWINGS

The invention will be better understood and features, aspects, and advantages other than those set forth above will become apparent when consideration is given to the following detailed description thereof. Such detailed description makes reference to the following drawings.

FIG. 1 is a schematic illustration of a human nervous system and identifiers of prospective sites and applications for nerve stimulation.

FIG. 2 is a schematic illustration identifying clinical challenges of nerve stimulation.

FIG. 3 is a schematic illustration of a nerve repair system for use in an open surgical approach, the nerve repair system including an electrotherapeutic nerve scaffold according to an embodiment of the disclosure.

FIG. 4 is a schematic illustration of a nerve repair system for use in an open or percutaneous surgical approach, the nerve repair system including an electrotherapeutic nerve scaffold according to an embodiment of the disclosure.

FIG. 5 is a schematic illustration of dissolution phases that occur during a percutaneous nerve repair surgery using the nerve repair system of FIG. 4.

FIG. 6 is a schematic illustration of electrodes being released during a percutaneous nerve repair surgery using the nerve repair system of FIG. 4.

FIG. 7 is a schematic illustration of a nerve repair system including an electrotherapeutic nerve scaffold according to an embodiment of the disclosure.

FIG. 8 is a schematic illustration of a nerve repair system including an electrotherapeutic nerve scaffold and an electrotherapeutic nerve gel according to an embodiment of the disclosure.

FIG. 9 is a schematic illustration of a nerve repair system including a nerve graft according to an embodiment of the disclosure.

FIG. 10 is a schematic illustration of a nerve repair system including a compound adhesive according to an embodiment of the disclosure.

FIG. 11a is an exemplary chart illustrating an optimization of acrylamide and alginate concentrations.

FIG. 11b is an exemplary chart illustrating an optimization of crosslinker concentrations.

FIG. 11c is an exemplary graph illustrating storage and loss moduli before and after dissolution to demonstrate weaking of an absorbable conductive electrotherapeutic scaffold following dissolution.

FIG. 11d is an exemplary graph illustrating a decrease in storage and loss moduli of a scaffold polymer over time.

FIG. 11e is an exemplary graph illustrating comparisons of various dissolution formulations.

FIG. 11f illustrates exemplary appearances of an absorbable conductive electrotherapeutic scaffold in a cuff embodiment at 0 and 4 weeks of incubation in saline.

FIG. 11g is an exemplary chart illustrating resistivity of an absorbable conductive electrotherapeutic scaffold.

FIG. 11h illustrates exemplary conformable embodiments of an absorbable conductive electrotherapeutic scaffold that can be extruded through particular needle gauges.

FIG. 12a illustrates an exposure of a sciatic nerve from an exemplary in vivo test of a nerve repair system including an electrotherapeutic nerve cuff.

FIG. 12b illustrates a transection of the sciatic nerve of FIG. 12a.

FIG. 12c illustrates a microsurgical coaptation of the sciatic nerve cuff of FIG. 12a.

FIG. 12d illustrates a surgical placement of the electrotherapeutic nerve cuff around a repair site of the sciatic nerve cuff of FIG. 12a.

FIG. 12e illustrates an adhesion and visualization of electrodes and leads of the nerve repair system of FIG. 12a.

FIG. 13a is an exemplary graph illustrating an electrical stimulation response of a tibialis anterior measured in response to proximal sciatic nerve stimulation during a test related to FIGS. 12a-e.

FIG. 13b is an exemplary chart illustrating a ratio of a sensory threshold in an affected versus unaffected limb for an electrical stimulation response.

FIG. 13c is an exemplary chart illustrating a ratio of mass of an affected versus unaffected gastrocnemius for an electrical stimulation response.

FIG. 13d is an exemplary chart illustrating a ratio of mass of an affected versus unaffected tibialis anterior for an electrical stimulation response.

FIG. 13e is an exemplary chart illustrating a maximum force required to remove electrodes of a nerve repair system.

FIG. 13f is an exemplary chart illustrating a ratio of distal versus proximal axon counts that were calculated in sural, peroneal, and tibial branches on an affected side compared to a contralateral side.

FIG. 13g is an exemplary image of a sciatic nerve descending into three branches of sural, peroneal, and tibial.

FIG. 14a is an exemplary image of adhesions on electrodes removed from a nerve repair site after an electrotherapeutic nerve cuff was dissolved.

FIG. 14b is an exemplary image of adhesions on electrodes removed from a nerve repair site after an electrotherapeutic nerve cuff was not dissolved.

FIG. 14c is an exemplary image of adhesions on electrodes removed from a nerve repair site after an electrotherapeutic nerve cuff was dissolved.

FIG. 14d is an exemplary image of adhesions on electrodes removed from a nerve repair site after an electrotherapeutic nerve cuff was not dissolved.

FIG. 15a is an exemplary image of an electrotherapeutic scaffold injected at 5 mm from a target nerve.

FIG. 15b is an exemplary image of an electrotherapeutic scaffold injected at 2.1 cm from a target nerve.

FIG. 15c is an exemplary image of an electrotherapeutic scaffold injected at 2.9 cm from a target nerve.

FIG. 15d is an exemplary graph illustrating a voltage amplitude versus a distance from a target area of a cervical vagus nerve.

15e is an exemplary graph illustrating a voltage amplitude versus a distance from a target area of a femoral nerve.

### DETAILED DESCRIPTION

The following discussion is presented to enable a person skilled in the art to make and use aspects of the present disclosure. Various modifications to the illustrated configurations will be readily apparent to those skilled in the art, and the generic principles herein can be applied to other configurations and applications without departing from aspects of the present disclosure. Thus, aspects of the present disclosure are not intended to be limited to configurations shown, but are to be accorded the widest scope consistent with the principles and features disclosed herein. The following detailed description is to be read with reference to the figures, in which like elements in different figures have like reference numerals. The figures, which are not necessarily to scale, depict selected configurations and are not intended to limit the scope of the present disclosure. Skilled artisans will recognize the non-limiting examples provided herein have many useful alternatives and fall within the scope of the present disclosure.

Peripheral nerve injury or dysfunction critically affects somatic and autonomic function, resulting in pain, immobility, and/or loss of functionality, which can significantly reduce quality of life in a patient. With over twenty million annual cases worldwide, peripheral nerve injuries result in an annual economic burden of $150 billion USD. However, less than half of cases yield satisfactory motor or sensory recovery after receiving standard medical care primarily due to poor axonal regeneration and disuse atrophy of distal muscles. Moreover, injuries and neuropathies occurring in autonomic nerves or in anatomically challenging locations, such as the celiac plexus, remain difficult to cure and are treated largely through non-specific pharmacotherapies.

Conventionally, peripheral nerve diseases are either treated in an open surgical (e.g., extremity nerve injuries, headache surgery) or minimally invasive fashion (e.g., spinal cord stimulators, percutaneous leads for pain management). However, neural interfacing and manipulation are inherently challenged by nerves' small dimension, delicate nature and often deep and complex anatomy adjacent to other critical structures. Further, device removal can be challenging and is associated with concerns for tissue trauma.

Electrical stimulation(ES) can accelerate axonal growth and therefore muscle regeneration with improved outcomes in the preclinical and clinical setting. However, broad translation to patients has not been possible with conventional methods given that practical long-term delivery of electrical stimulation with devices has been challenging.

In some conventional methods, during open surgery, electrodes can be precisely placed around the affected nerve. However, practical and precise placement of the device, mechanical interference with the nerve repair, fibrotic adhesion of the device to the nerve, and eventual removal without tissue trauma or a secondary surgery are conventionally difficult and largely unsolved barriers. Currently, nerve surgeons may use nerve cuffs made out of decellularized grafts to prevent axonal sprouting (diffuse nerve regeneration) and protect peripheral nerve repairs. However, there is no added therapeutic benefit for the patient. Combining a nerve cuff that displays all the practical attributes for successful clinical implementation (e.g., practical placement, absorbable, atraumatic electrode removal) with electrostimulation would be readily accepted by surgeons and electrotherapy would enhance the nerve repair leading to improved outcome.

While some advances in minimally invasive techniques including laparoscopy, robotics or interventional radiology-based percutaneous approaches have made it possible to access deep-set nerves in the body, it remains difficult to place leads on target millimeter caliber nerves with the millimeter precision required to prevent off-target effects. Further, lead migration is a highly common complication with many reports quoting rates between 60-100%, requiring repeated operations. An electrotherapeutic gel with adherent electrodes that can be precisely placed in the required location without migration and dissolved when no longer needed would be beneficial for many clinical applications such as spinal cord stimulation, and autonomic dysfunction (e.g., bladder dysmotility, gastroparesis, pelvic dysfunction).

In general, nerve stimulation has shown promise in applications ranging from peripheral nerve regeneration after injury to therapeutic organ stimulation. However, as briefly discussed above, clinical implementation has been impeded by various technological limitations, including surgical placement, lead migration, and atraumatic removal.

In some instances, electrical peripheral nerve stimulation (PNS) has been clinically demonstrated to significantly improve sensory and motor function in contexts where surgery alone has had limited effectiveness. Following nerve crush, transection or stretch injury, PNS cam accelerate axonogenesis by modulating plasticity, elevating neuronal cyclic adenosine monophosphate, and upregulating neurotrophic factors and Schwann cell activity. Chronic PNS prevents disuse atrophy of distally innervated muscles by reducing apoptosis of denervated muscle fibers, which preserves muscle mass. Moreover, for autonomic conditions of neural etiology, such as incontinence, sleep apnea, and gastrointestinal motility, PNS can restore and even augment function. Despite the widespread and evolving evidence of the benefits of PNS, its clinical implementation has been limited to treating intractable pain pathologies such as lower back pain and occipital neuralgia; little to no clinical translation of PNS has occurred for peripheral nerve repair or for non-pain indications in autonomic nerves due to hardware implantation challenges.

Conventionally, leads for peripheral nerves can be placed using an open surgical approach (e.g., to treat extremity nerve injuries, or for migraine surgery), while autonomic nerves deep in the body (e.g., in the celiac plexus, spinal cord, or SMA plexus adjacent to the aorta) can be accessed through minimally invasive percutaneous approaches including laparoscopy, robotics, and interventional radiology. Placement of nerve stimulation hardware is complicated by the need to precisely target millimeter-sized nerves to prevent off-target stimulation, achieve lead stabilization on delicate neural tissue, and avoid mechanical interference with nerve repairs.

As briefly discussed above, following implantation, lead migration is a common complication, with 60-100% of cases requiring subsequent re-operation. Atraumatic removal is complicated by tissue adhesion to the leads. Consequently, at the completion of therapy or when the device malfunctions, leads are frequently left in the body, posing potential discomfort, and serving as a nidus for infection. Some systems can require in vivo free radical polymerization which can present a potential carcinogenic risk. As further discussed throughout the present disclosure, a conformable scaffold capable of stabilizing leads, bridging conduction gaps between probes and target nerves, and releasing hardware on demand for atraumatic removal is provided, for example for the clinical implementation of PNS or other applications.

One conventional standard-of-care involves nerve conduits or nerve wraps that are sewn together to mechanically hold both ends of the injured nerve in place. These can consist of three-dimensional cylindrical structures made of a decellularized human nerve allograft. While these might be able to preserve the inherent structure of the extracellular matrix (ECM), they provide no targeted acceleration of nerve regeneration. These are primarily used to relieve tension at the coaptation site, aid in grafting or cable grafting repairs, and reinforce the coaptation site. Furthermore, while some studies have demonstrated the positive benefit of locally delivering growth factors or immunomodulators, conventional methods do not locally dose a site of nerve repair. Thus, there is a need for systems and methods that address these and other drawbacks.

Systems and methods are provided to facilitate structural stabilization, for example, of a nerve. Furthermore, system and methods are provided to remove the structural stabilization without traumatizing the structure, such as a nerve, that is the focus of the stabilization. Additionally, systems and methods are provided herein related to a nerve conduit capable of temporal or permanent electrical stimulation with the capability to easily release embedded hardware or impregnated therapeutic molecules. In one example, a nerve conduit system is provided and a method of using such a nerve conduit is provided, among other embodiments and concepts.

In one example embodiment, a nerve conduit may be provided. The nerve conduit can include one or more of a mechanical support for a nerve repair site; embedded stimulation contacts or probes to deliver stimulation (such as electrical or optical) to the target site of nerve repair and to distal muscles innervated by the target nerve; a polymer formulation with adhesive and fast, triggerable dissolution properties thereby enabling damage-free removal of hardware; and/or a conductive polymer formulation. The conductive polymer formulation may be configured to enhance stimulation, distribute stimulation pulses to an entire region of contact, and/or enable bridging a stimulation contact to a target site.

Furthermore, in some embodiments, a nerve conduit can include one or more of drug eluting properties to enable localized delivery of therapeutic agents including gene-based therapies, a microfluidic catheter that enables localized release of therapeutic agents, and/or a microfluidic catheter that enables localized sampling of extracellular fluid. Additionally, in some embodiments, a nerve conduit can provide a formulation as an injectable composition.

In some embodiments, a nerve conduit can include a polymer gel that can be dissolved upon injection of a solution and may contain a mesh support. For example, a mesh support may include a knitted mesh, for example, a Vicryl mesh. However, any of a variety of other types of absorbable materials can be used (mesh and non-mesh), such as Polyglactin and Polyglycolic-based materials. The nerve conduit may be comprised of an interpenetrating network (IPN) alginate poly-acrylamide hydrogel. The nerve conduit can control against adhesions directly to the nerve that would interfere with nerve regeneration, hardware implantation, scarring, and signal-to-noise ratios of electrophysiology performed through the contacts. Further, if electrodes are included, adhesion directly to the electrodes is likewise controlled against, therefore, allowing for atraumatic removal.

Some embodiments of the disclosure provide a nerve conduit or graft can house a set of electrodes or other probes that are placed on either side of the repair, as well as a secondary gel layer that can be loaded with biologic APIs. The proximal electrode and distal electrode are activated in a sequence that enables axonogenesis. The distal electrode can conduct a signal that stimulates the muscle or end organ innervated by the nerve to prevent atrophy. Though electrical stimulation is described in detail herein, optical probes designed to deliver optical stimulation to the nerve may be used, as an alternative to or in addition to any electrical contacts and electrical stimulation.

Generally, embodiments of the disclosure can provide an absorbable conductive electrotherapeutic scaffold (ACES). In some embodiments, the ACES can include an alginate/poly-acrylamide-based interpenetrating network (IPN) hydrogel impregnated with gold nanoparticles. The scaffold can facilitate placement and stabilization of leads at target sites by mechanically conforming to the surrounding anatomical features. ACES can be used to restrict granulation and scar formation to the exterior of a dissolvable gel cavity, rather than around the electrodes, to maximize stimulation efficacy and minimize trauma upon removal.

When therapeutic stimulation is complete and the hardware is to be removed, the scaffold can be triggered to dissolve, facilitating atraumatic removal. ACES can be designed in application-specific embodiments. For nerve repairs utilizing an open surgical approach, ACES can be pre-formed into a cuff, situating one electrode each on the proximal and distal sides of a nerve repair site to provide electrical stimulation(ES) for axonogenesis and distal organ stimulation.

Additionally or alternatively, a grounded-gel formulation of ACES can be applied for percutaneous and open surgical approaches. This formulation can be injected at the site of the nerve, where it stabilizes electrodes, conforms to the surrounding anatomical features, and establishes a conductive path from the leads to the target nerve without requiring direct electrode-nerve contact. Upon triggered dissolution through a co-implanted microcatheter or injection, electrodes can be released from the gel into the cavity of the scaffold and can be removed transcutaneously in an atraumatic fashion. The gel's mechanical properties can be matched to that of the nerve to permit nonintrusive support, prevent rejection, and provide mechanical support, accelerating axonogenesis.

In some embodiments, ACES can include an alginate/poly-acrylamide interpenetrating network hydrogel optimized for both open and minimally invasive percutaneous approaches. In one example of a model of sciatic nerve repair, ACES significantly improved motor and sensory recovery (p<0.05), increased muscle mass (p<0.05), and increased axonogenesis (p<0.05). Triggered dissolution of ACES can enable atraumatic, percutaneous removal of leads at forces significantly lower than controls (p<0.05). In one example of a porcine model, ultrasound-guided percutaneous placement of leads with an injectable ACES near the femoral and cervical vagus nerves facilitated stimulus conduction at significantly greater lengths than saline controls (p<0.05). Overall, ACES can facilitate lead placement, stabilization, stimulation and atraumatic removal enabling therapeutic PNS.

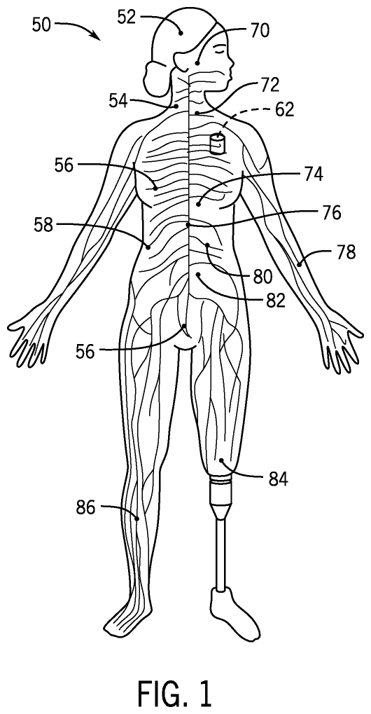

Referring now to FIG. 1, a schematic of a human nervous system 50 is shown. Conventionally, peripheral nerve stimulation (PNS) applications can be used in the treatment of occipital neuralgia (e.g., headaches) 52, cervical radiculitis 54, intercostal neuralgia 56, lumbar radiculitis (e.g., lower back pain) 58, pudendal neuralgia, cluneal nerve pain, meralgia paresthetica, and lower extremity neuropathies 60, for example. Additionally, PNS can also be used during the implantation of a pulse generator 62.

According to embodiments of the present disclosure, nerve stimulation can further advantageously be used, for example, in facial nerve stimulation 70, laryngeal nerve stimulation 72, diaphragmatic stimulation 74, treating paralysis 76, peripheral nerve repair 78, gastric and hepatic stimulation 80, colonic motility 82 (e.g., via vagal modulation), treating phantom limb pain 84, and treating urinary incontinence 86 (e.g., via posterior tibial nerve stimulation).

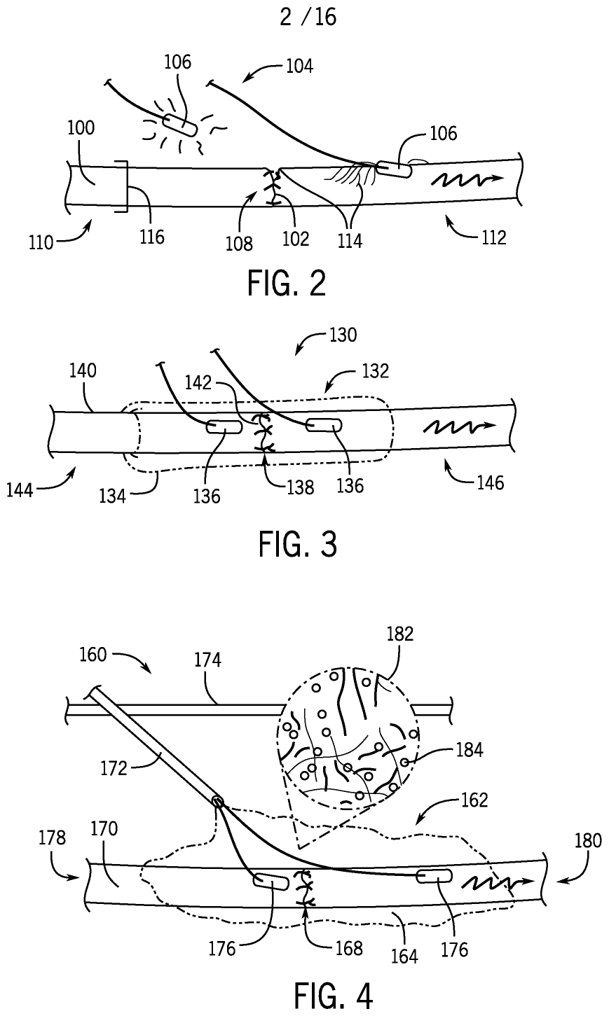

With reference to FIG. 2, clinical challenges associated with peripheral nerve stimulation will be described. Some conventional techniques used to repair a nerve 100 having a nerve tear 102 can include the use of implanted peripheral nerve stimulation hardware 104. The hardware 104 can include first and second electrode leads 106 that can be placed on either side of the nerve repair site 108 so that the nerve tear 102 is between the electrode leads 106 and one lead 106 is arranged on a proximal end 110 of the nerve 100 and the other lead 106 is arranged on a distal end 112 of the nerve 100. Specific challenges associated with using such conventional nerve stimulation hardware 104 include lead 106 migration, scarring 114, precise targeting of millimeter caliber 116 nerves, and overall traumatic removal.

Embodiments of the invention can address these and other drawbacks. For example, FIG. 3 illustrates an example of a nerve repair system 130 according to an embodiment of the disclosure. The nerve repair system 130 can be used in an open surgical approach and can include a scaffold 132. The scaffold 132 can be configured as an electrotherapeutic nerve scaffold. In particular, the scaffold 132 can be configured as an absorbable conductive electrotherapeutic scaffold (ACES).

In the illustrated embodiment, the nerve repair system 130 can include a cuff 134. A pair of electrodes 136 can, optionally, be included. In use, the cuff 134 can be deployed around a nerve repair site 138 of a nerve 140. As shown in FIG. 3, the nerve repair site 138 includes a nerve tear 142, however, other types of nerve repair sites are possible. The electrodes 136, if included, can then be secured relative to the nerve 140 with one electrode 136 positioned at a proximal side 144 of the nerve repair site 138 and the other electrode 136 positioned at a distal side 146 of the nerve repair site 138. The electrodes 136 can then be used to propagate stimulation to the distal muscle near the repair site 138.

With reference now to FIG. 4, another example of a nerve repair system 160 according to an aspect of the disclosure is shown. The nerve repair system 160 can be used in open or percutaneous surgical approaches and can include a scaffold 162. In the percutaneous surgical approach, the procedure can be minimally-invasive. In use, the scaffold 162 can be deployed as a stabilizing material 164. The stabilizing material may be formed of a substance or material formed of macromolecules forming repeating subunits, or polymer. More particularly, a polymer may include a protein or polysaccharide. Further, the stabilizing material may include at least one of a polymer, protein, polysaccharide, metal, lipid, ceramic. The stabilizing material may include a material that is configured to degrade upon being subjected to the dissolution solution without damaging the in vivo nerve. In one example, the polymer may be a gel or other material that is suitable to be arranged or injected percutaneously around a nerve repair site 168 of a nerve 170 where the stabilizing material 164 conforms to the surrounding anatomy of the nerve 170. The stabilizing material 164 can be injected via a needle 172 or via other means that can be inserted at a skin surface 174 into deep tissue. For example, the stabilizing material 164 may be shaped to a cylindrical mold or format. It may be comprised of a viscosity to be injectable. It may be formed of an adjustable concentration and/or density to make it mechanically compliant to the same degree as the nervous tissue. It may incorporate gold nanoparticles or other conductive ions to modulate conductivity for electrical stimulation applications. In addition, it is contemplated that one may titrate the concentrations so as to prevent neural cell toxicity. Further, it may incorporate leads with the appropriate trigger electronics to induce dissolution, as will be described. In one example, the stabilizing material 164 can be configured as an electrotherapeutic nerve gel (ENG).

The nerve repair system 160 can further include first and second electrodes 176. The scaffold 162 formed by the stabilizing material 164 can stabilize and/or adhere to the electrodes 176. The electrodes may be placed near the nerve repair site 168, such as at proximal 178 and distal sides 180 of the nerve repair site 168. The electrodes 176 can then be used to propagate stimulation to the distal muscle near the nerve repair site 168. The scaffold 162 can conduct stimulation signals from the leads 176 to the nerve 170. In general, the gel 164 can bridge a conductivity gap between stimulation hardware (e.g., the electrode leads 176) and the target nerve 170.

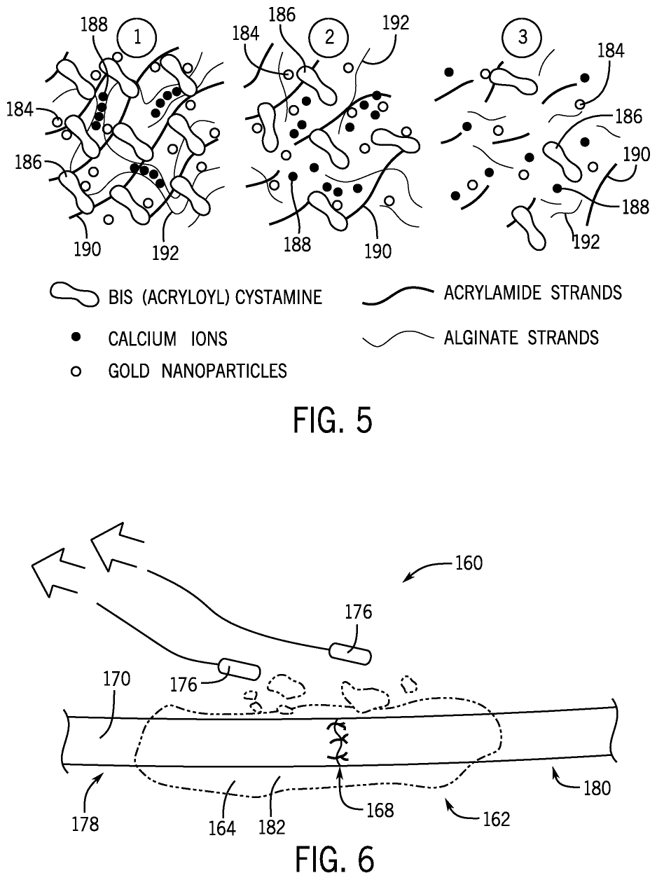

In some embodiments, the stabilizing material 164 can include a shredded formulation 182 that can include gold (Au) nanoparticles 184. In use, once the stimulation treatment has concluded, a dissolution event can be triggered (e.g., ACES dissolution) to begin an electrode removal process. As depicted in FIG. 5, dissolution can occur through chelation of calcium releasing cross-links. In some embodiments, the dissolution can include the separation of a disulfide (e.g., bis (acryloyl) cystamine) 186, calcium ions 188, gold nanoparticles 184, acrylamide strands 190, and alginate strands 192. In other configurations, the dissolution material may include light designed to trigger dissolution of the stabilizing material. In this way, the light may be delivered via LEDs, either externally or arranged in the electrode leads.

With reference to FIG. 6, once the scaffold 162 is cavitated (e.g., via the dissolution process), the electrode leads 176 can be atraumatically removed from the nerve repair site 168 of the nerve 170 with minimal force. In particular, the leads 176 can be removed without trauma such as ripping, scarring, pulling, straining, etc. the nerve 170 at or around the nerve repair site 168. Additionally, once the electrodes 176 are removed from the dissolved scaffold 162 cavity, the stabilizing material 164 can be allowed to absorb, dissipate, and/or dissolve so that additional surgical intervention is not required to clear the stabilizing material 164 from the patient.

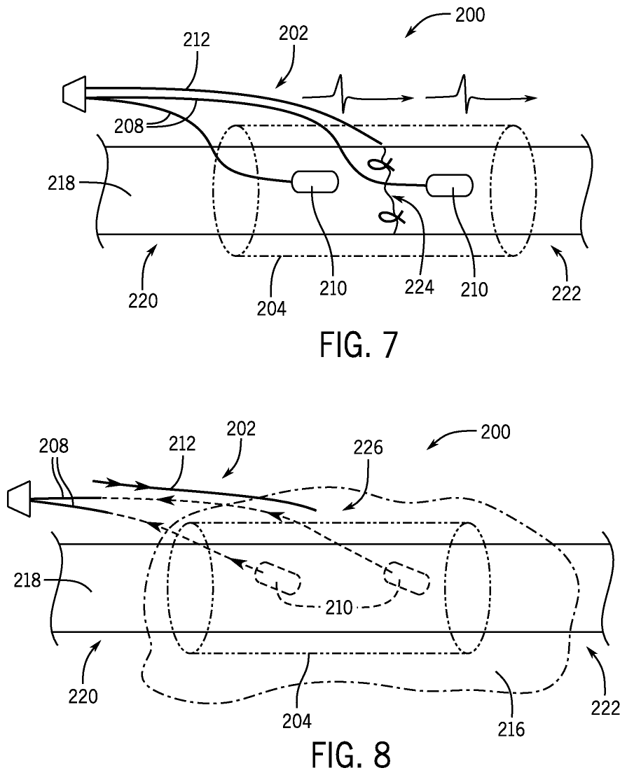

FIGS. 7 and 8 illustrate another example of a nerve repair system 200 according to the disclosure. The nerve repair system 200 in an electrotherapeutic nerve scaffold 202 having a nerve cuff 204. In the illustrated embodiment, the electrotherapeutic nerve scaffold 202 can be configured as an absorbable, electrotherapeutic nerve scaffold (ENS) and the nerve cuff 204 can be configured as an absorbable electrotherapeutic nerve cuff (ENC). In general, the electrotherapeutic nerve scaffold 202 can allow for practical placement of the nerve cuff 204 during an open surgical approach. The nerve repair system 200 can further include electrodes 208 having electrode leads 210. Additionally, in some embodiments, the nerve repair system 200 can include a microcatheter 212.

Various nerve repair systems described herein, including the nerve repair system 200, can include a stabilizing material 216, which may be a gel such as an interpenetrating network (IPN) alginate poly-acrylamide hydrogel. Such gel can allow for a scaffold, such as the electrotherapeutic nerve scaffold 202, to permit tissue healing around its surface, rather than around electrodes and electrode leads. This feature can advantageously prevent adhesions directly to a nerve (e.g., the nerve 218 of FIGS. 7 and 8) which can interfere with nerve regeneration. Further, with reference to FIGS. 7 and 8, the electrode leads 210 are not adhered directly to the nerve 218, therefore there is no atraumatic nerve removal.

In use, a cuff of a nerve repair system 200, such as the nerve cuff 204 of FIGS. 7 and 8, can be a stabilizing material 216 that is preformed into a cuff-like geometry. The cuff 204 can allow one of each electrode lead 210 to be placed on proximal 220 and distal 222 sides of a nerve repair site 224. The electrodes 208 and electrode leads 210 can provide electrical stimulation for axonogenesis and distal organ stimulation. Distal muscle stimulation can prevent denervation atrophy. In a minimally-invasive approach (see, for example, FIG. 4), a shredded formulation of the gel (ENG) can be injected percutaneously to the site of a nerve, where it conforms to the surrounding anatomy.

In use, a microcatheter of a nerve repair system, such as the microcatheter 212 can be used to trigger dissolution 226 (see, for example, FIG. 8). In other embodiments, an injection can be used for dissolution (e.g., triggered dissolution). During (or after) the dissolution process, the electrode leads 210 can be released into a cavity of the electrotherapeutic nerve scaffold 202 and can be removed from the patient. According to embodiments of the disclosure, a gel used in a nerve repair system, such as the stabilizing material 216, can include mechanical properties that match (or are substantially medically similar) to that of the nerve 218 to facilitate surgical repair and provide mechanical support to accelerate axonogenesis. Additionally, as described above, gold nanoparticles (Au NPs) can be incorporated into the gel to increase gel conductivity and bridge distances between targe nerves and stimulation hardware.

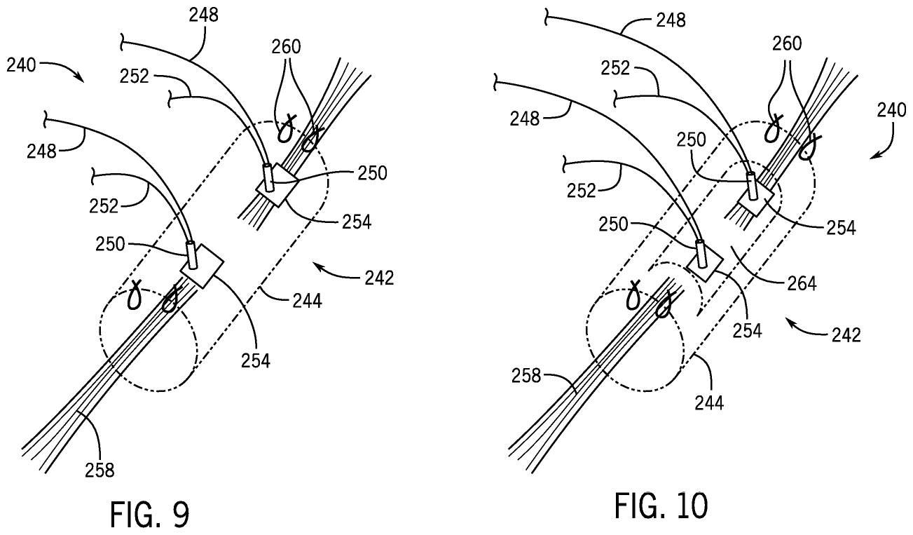

FIGS. 9 and 10 illustrate another example of a nerve repair system 240 according to the disclosure. The nerve repair system 240 can include a scaffold system 242, a nerve graft 244, a pair of electrode wires 248 having electrode leads 250, and a catheter 252. Additionally, in the illustrated embodiment, the nerve repair system 240 can include an electrode adhesive 254. In some embodiments, the electrode adhesive 254 can be adhered to or sutured to the nerve graft 244. The electrode adhesive 254 can be used to secure the electrode leads 250 relative to the nerve 258 and graft ligatures 260 can be used to secure the graft 244 relative to the nerve 258.

In the illustrated embodiment, each of the nerve graft 244 and the electrode adhesive 254 may be conductive while also absorbable or dissolvable by the patient. In some embodiments, the graft ligatures 260 may similarly be absorbable or dissolvable. Additionally, the electrode adhesive 254 can include an interpenetrating network of hydrogel. In addition or alternatively to the electrode adhesive 254, the nerve repair system 240 can include a biocompatible polymer 264, such as an alginate N-hydroxysuccinimide ester for adhesion.

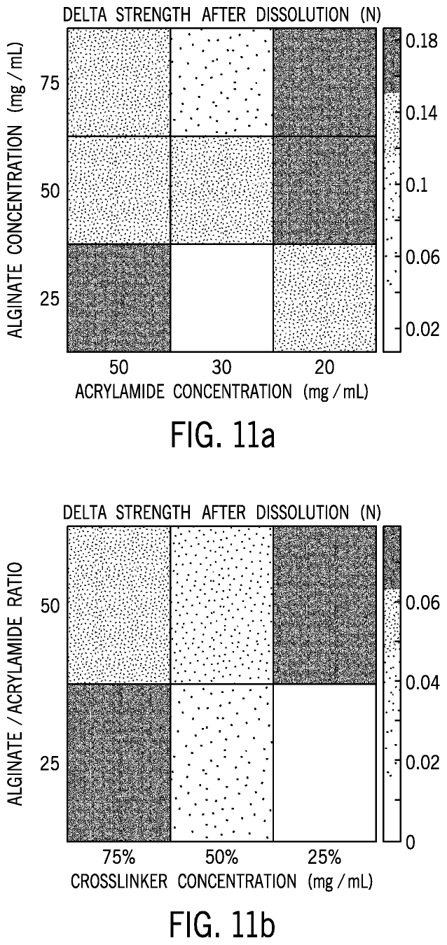

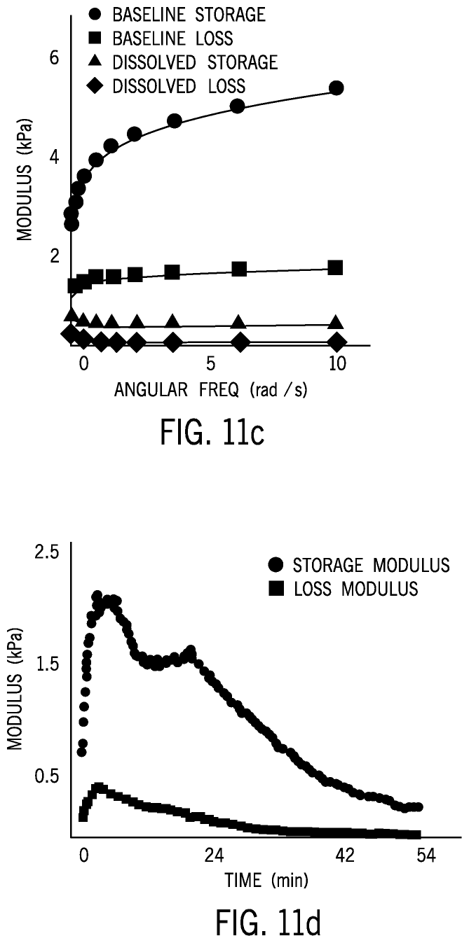

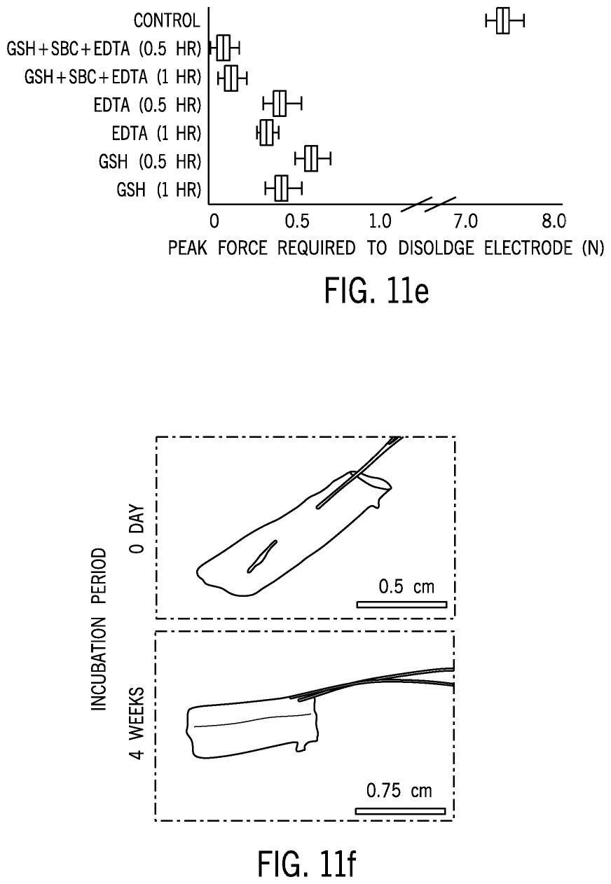

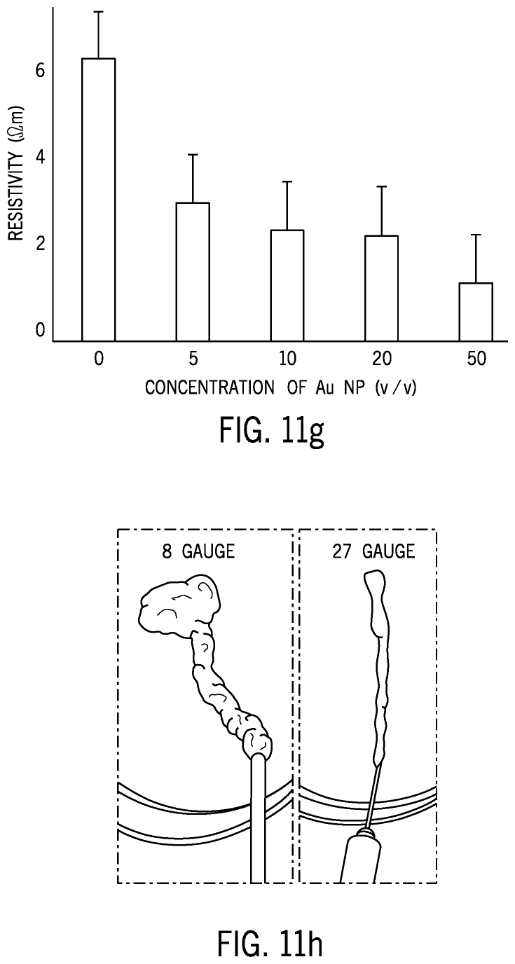

FIGS. 11-15 illustrate exemplary results of studies performed using embodiments and configurations of nerve repair systems described herein. One exemplary study tested the efficacy of an electrotherapeutic nerve cuff (ENC) device, similar to the scaffold 132 of FIG. 3, the nerve cuff 204 of FIGS. 7 and 8, and the scaffold system 242 of FIGS. 9 and 10. The ENC device was designed using knitted polyglactin 910 mesh, and two microelectrodes coated in dissolvable, electroconductive inert gel. Gel strength was optimized by maximizing the release force of an electrode while maintaining extrudability and non-toxicity under ISO norms.

In general, FIGS. 11a-h illustrate an exemplary optimization of a triggerable polymeric formulation for an absorbable conductive electrotherapeutic scaffold (ACES). FIGS. 11a and 11b illustrate measurements that represent the average of three independent samples. FIG. 11c illustrates storage and loss module before and after dissolution. FIG. 11d illustrates a time course of dissolution that demonstrates a monotonal decrease in the storage and loss moduli of a scaffold polymer. The storage modulus for the immersed sample ends up ˜60% of the pre immersion sample and approximately 250% of the 24 hr degraded sample, indicating that after 66 mins the sample is 40% degraded and 2.5× the minimum strength.

To enable optimal tissue adherence and trauma-free removal of leads, the exemplary study used a tough, adhesive polymer that could undergo a significant decrease in strength after dissolution. Covalently crosslinked polyacrylamide was selected for its adhesive and elastic properties while ionically crosslinked alginate was selected for its flexibility and triggerable weakening. To enable triggerable dissolution of the polyacrylamide network, N,N′-Bis (acryloyl) cystamine was chosen as the covalent crosslinker due to its labile disulfide bond.

To optimize the interpenetrating network (IPN) formulation, various combinations of acrylamide, alginate, and crosslinker concentrations were evaluated for adhesive strength and subsequent dissolution capacity through an electrode removal assay. The elastic modulus of the gel was tuned to be comparable to peripheral nerve (772.8±244.3−4387.6 Pa24). A ratio of 50 mg/mL acrylamide and 25 mg/mL alginate with 75% crosslinker concentration resulted in the greatest reduction in strength after dissolution (see FIGS. 11a and 11b). Upon adhesion of the IPN to the electrodes, the leads withstood up to 4.96+/−0.08 N of tensile force with no measurable displacement. Dissolution was initially performed with 0.5 M reduced glutathione (GSH), which cleaves the labile disulfide bond within the bis (acryloyl) cystamine crosslinker, and EDTA (Ethylenediaminetetraacetic acid), which chelates Ca2+ ions and thus cleaves the ionic alginate crosslinks.

Upon dissolution, the electrodes slid out with 2.3+/−0.03 N of force. To evaluate the time-dependent viscoelastic behavior, an oscillatory shear test was performed before and after the dissolution of ACES (see FIG. 11 c). The storage and loss moduli significantly decreased following dissolution (>3 fold, p<0.01, Student's t-test). The reduction in loss modulus reflects the dissolved alginate crosslinks, which allow the leads to slide out with little frictional force. To determine the optimal incubation time for dissolution, the storage and loss moduli of the polymer under immersion for a period of 24 hours was rheometrically characterized.

At 30 minutes, an 85% reduction in storage modulus and 98% reduction in loss modulus was observed (see FIG. 11d). Within 1 hour, polymer weakening was comparable to that of 24 hours. Thus, 60 minutes was determined to be sufficient for dissolution. To further optimize the dissolution profile, a variety of formulations of GSH, SBC (sodium bicarbonate), and EDTA at 30- and 60-minute periods using an electrode removal assay were studied. All dissolution treatments enabled electrode removal at the pre-strain threshold of the tensile test (0.01N), requiring no additional force to pull out the electrode (see FIG. 11e). In contrast, in controls with no treatment, 7.62N of force was required to pull out the electrode—an over 750-fold increase in force. A solution of 0.5M GSH+0.5M SBC+1M EDTA was selected for its ability to effectively dissolve the ACES. Thus, embodiments of the disclosure advantageously provide systems and methods that can reduce the force required to remove electrodes during a nerve repair procedure.

For open surgical approaches, a preformed cuff-like scaffold shape provides mechanical support and eases surgical placement around the nerve. Thus, ACES was preformed into a custom mold, incorporating electrodes and a microfluidic channel to deliver the dissolution reagents (see FIG. 11f, top). Following a four-week incubation in saline at 37° C. to mimic the tissue environment, no significant degradation, tearing or disintegration was observed (see FIG. 11f, bottom). Furthermore, the ACES's modulus and electrode removal assay results before and after incubation demonstrated no significant differences (p<0.05, Student's two-tailed heteroscedastic t-test, n=4 trials).

As described above, FIGS. 11a-h generally illustrate an optimization study of a triggerable polymeric formulation for an absorbable conductive electrotherapeutic scaffold. In particular, FIG. 11a represents an optimization of acrylamide and alginate concentrations which yielded the greatest differential strength in the 50 mg/mL acrylamide and 25 mg/mL alginate formulation. FIG. 11b illustrates an optimization of crosslinker concentrations which yielded the greatest differential strength with 75 μg/mL for a 25%. FIG. 11c illustrates storage and loss moduli before and after dissolution to demonstrate a significant weaking of the ACES following dissolution. FIG. 11d illustrates a time course of dissolution which demonstrates a monotonal decrease in the storage and loss moduli of the scaffold polymer. FIG. 11e illustrates a comparison of various dissolution formulations. FIG. 11f illustrates an appearance of the ACES in a cuff embodiment at 0 and 4 weeks of incubation in saline. FIG. 11g illustrates a resistivity of ACES formulations. FIG. 11h illustrates a conformable embodiment of ACES that can be extruded through a range of needle gauges from 8-34. For FIGS. 11a-h include measurements that represent the average of independent samples.

In the study represented by FIGS. 11a-h, to bridge the conductivity gap between stimulation hardware and the target nerve in minimally invasive surgical approaches, the conductivity of the scaffolding material was increased by incorporating gold nanoparticles (Au NPs, 40 nm), selected for their biocompatibility and conductivity. Compared to ACES with no added Au NPs, sheet resistance decreased 5-fold with an Au NP concentration of 5.5×1013 particles/mL as per a four-point contact test (see FIG. 11g). The gel's self-healing properties were leveraged to create an injectable embodiment. After polymerizing ACES as a 2 mm sheet, thin 2-4 mm strips were cut and loaded into a syringe. Injectability was then determined using extrusion equations. Given the gel's consistency index of K=21.996 Pa*sn and a shear-thinning index (n) of 0.134, it was determined that the gel could be injected through needles ranging from 7-34 gauge under a pressure of 2.6 MPa (human force of 50 N on a 5 mm diameter plunger) using the power-law fluid equations.

In one exemplary study, a biocompatibility of an ACES system was evaluated using an extract exposure test at treatment concentrations between 50-200 mg/mL and with varying Au NP concentrations at 1 and 7 days. Cell viability normalized to the vehicle treatment group was greater than 100% after 24 hours and greater than 70% after 7 days, which is considered non-toxic by ISO 10993 norms.

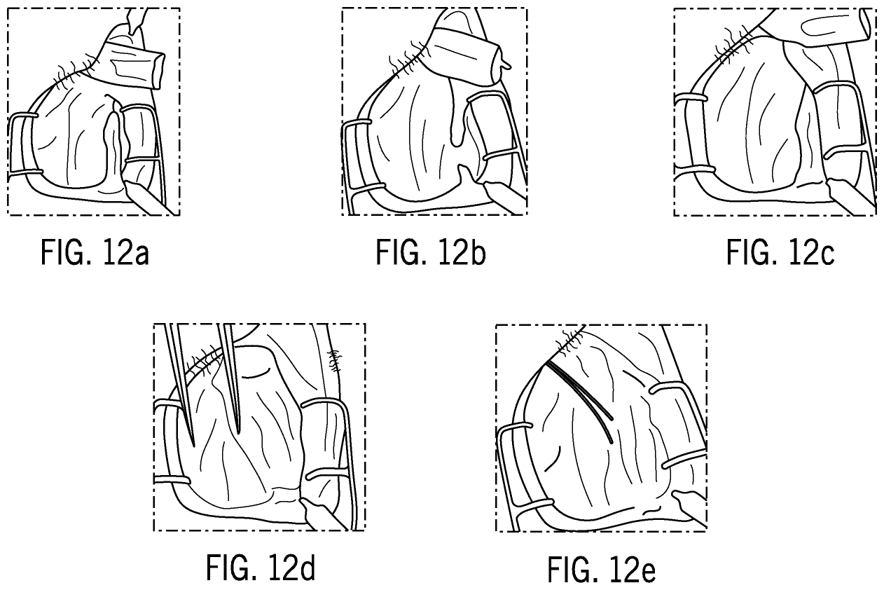

FIGS. 12a-e illustrate other exemplary results of studies performed using embodiments and configurations of the nerve repair systems described herein. In one exemplary study, thirteen Sprague Dawley rats (six experimental, seven controls) underwent unilateral surgical transection of the sciatic nerve with immediate microsurgical repair, as well as implantation of an electrotherapeutic nerve cuff (ENC) device.

The surgical process in the exemplary studied included exposure of the sciatic nerve (FIG. 12a), transection of the sciatic nerve (FIG. 12b), microsurgical coaptation of the sciatic nerve (FIG. 12c), surgical placement of ENC around repair site (FIG. 12d), and adhesion and visualization of the electrodes and leads in the scaffold (FIG. 12e).

The experimental group was stimulated every other day for 6 weeks in 30-minute sessions delivering current with a 20 Hz frequency, 2 mA amplitude, and 100 μs pulse width, whereas the control group was not stimulated. Outcome variables were recorded at baseline and weekly after surgery for a total of 6 weeks. Motor function variables included muscle electrophysiology (EMG) using the Intan RHS hardware and RHS software, sciatic functional index, as well as wet muscle weight and axon counts proximal and distal to the site of scaffold implantation after animal sacrifice. Sensory function was evaluated with a cutaneous sensitivity test (Bioseb calibrated forceps). Immediately after euthanasia at 6 weeks postoperatively, the experimental animals were injected percutaneously with dissolution solution at the site of the ENC, whereas the control group was not injected. The electrodes were removed using the Instron tensile testing machine and the release force was measured.

In another exemplary study, the feasibility of placement of the electrotherapeutic nerve gel (ENG) in a minimally-invasive manner in a porcine model was studied. Ultrasound-guided placement of the ENG at the femoral and cervical vagus nerves was performed. A 14 gauge needle was advanced to the target nerve, visualized using a linear ultrasound probe (Sonoscape S9 portable ultrasound system, Model ST-180) at a depth of 4 inches. 3 mL of the ENG material or saline (control) was injected. Then, an electrode was advanced through the needle and placed at a measured distances from the nerve. Electrophysiology of the biceps femoris or distal vagus nerve were performed using 2 bipolar 32-gauge needle electrodes (Natus Medical). Stimulation was performed using a model 2100 isolated pulse stimulation (A-M Systems) at 2 Hz, 20 pulses, 2-6 mA.

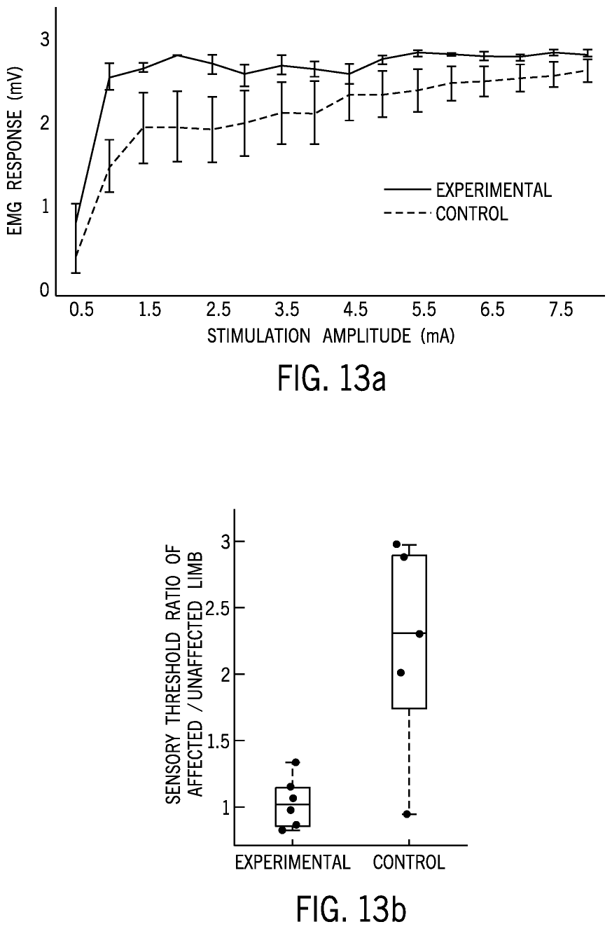

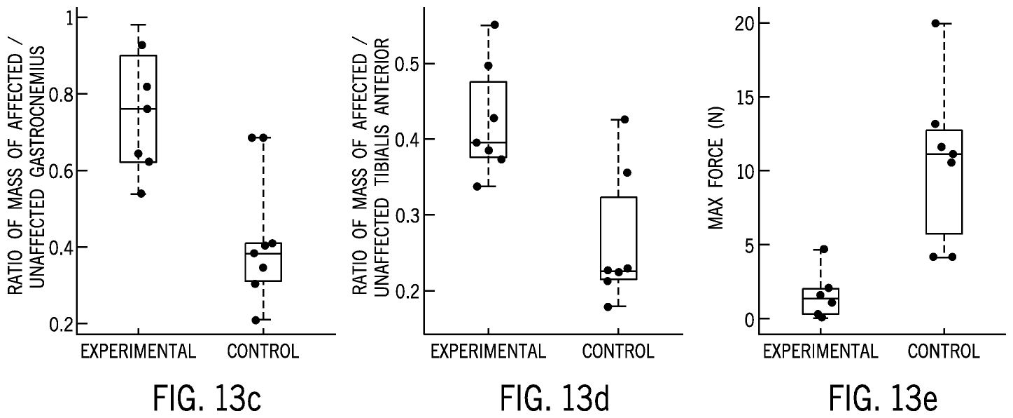

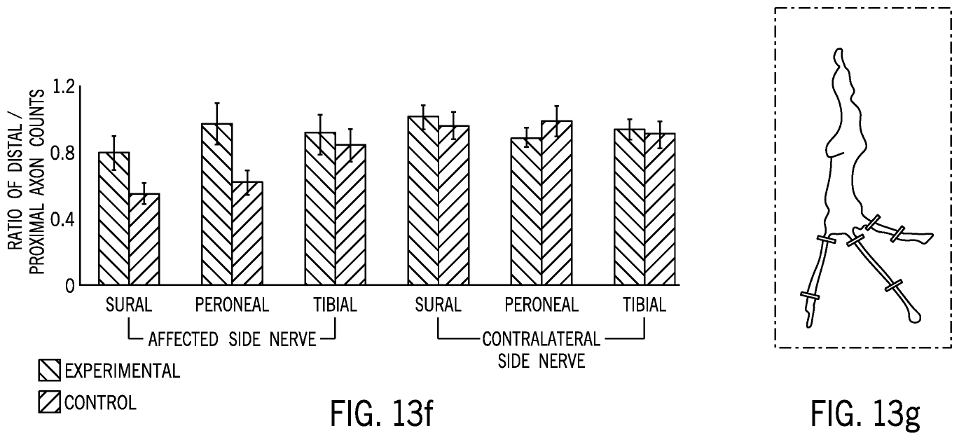

FIGS. 13a-g illustrate additional exemplary results of studies performed using embodiments and configurations of the nerve repair systems described herein. In particular, FIGS. 13a-g an exemplary electrotherapeutic nerve cuff (ENC) study. The study represented in FIGS. 13a-g may correspond to the stud represented in FIGS. 12a-e. As shown in FIG. 13a, muscle electrophysiology (EMG) responses were significantly higher, whereas the EMG activation threshold was significantly lower in the experimental group as compared to the control group at six weeks (p<0.05, student's t-test). As shown in FIG. 13b, sensory recovery returned to baseline in the experimental group as compared to the control group at six weeks (p<0.06). As shown in FIGS. 13c-e, wet muscle mass was significantly higher in the experimental group as compared to the control group (p<0.05, student's t-test). As shown in FIG. 13f, axon count ratios in the sural and peroneal nerve on the affected side were significantly higher in the experimental group compared to the control group.

As shown in FIG. 13a, EMG response of a tibialis anterior was measured in response to proximal sciatic nerve stimulation via the ENC electrodes resulting in a significantly higher EMG response in the experimental group than the control at stimulation amplitudes between 1-3.5 mA. As shown in FIG. 13b, a ratio of the sensory threshold in the affected to unaffected limb in the control group was significantly higher than the experimental group than the control group (p<0.05). As shown in FIG. 13c, the ratio of mass of the affected to unaffected gastrocnemius was significantly higher in the experimental group than the control group (p<0.05). As shown in FIG. 13d, the ratio of mass of the affected to unaffected tibialis anterior was significantly higher in the experimental group than the control group (p<0.05). As shown in FIG. 13e, the max force (N) needed to remove the electrodes was greater in the control group than the experimental group. As shown in FIG. 13f, the ratio of the distal to proximal axon counts were calculated in the sural, peroneal, and tibial branches on the affected side compared to the contralateral side. The experimental group had a significantly higher ratio in the sural and peroneal branches on the affected side. FIG. 13g is an exemplary image of the sciatic nerve descending into the three branches of sural, peroneal, and tibial.

Referring back to FIGS. 12a-e, in all groups, the sciatic nerve was transected, acutely repaired, and supported by the ACES in either cuff or injectable embodiments, requiring less than 5 or 2 minutes for stable placement, respectively. Over the course of 6 weeks, electrical stimulation applied at the sciatic nerve resulted in selective activation of the hind limb; no significant change in electrode impedance was observed, suggesting no lead migration in animals with ACES. Six weeks postoperatively, compound muscle action potentials (CMAPs) in the experimental group were significantly higher than untreated controls (p<0.05, Student's two-tailed heteroscedastic t-test) with a significantly lower stimulation-response threshold (p<0.05, Student's two-tailed heteroscedastic t-test).

The sensory thresholds in the control animals were significantly higher in the affected limbs compared to the contralateral unaltered limbs (p<0.05, Student's two-tailed heteroscedastic t-test), suggesting incomplete sensory reinnervation. In contrast, the sensory threshold ratio between the affected limbs and unaltered limbs in the experimental group was 1.02, suggesting a full sensation recovery in the limb supported by the ACES-based rehabilitation. The sensory thresholds of the contralateral unaffected sides of animals in each group were insignificantly different (p<0.001, Student's two-tailed heteroscedastic t-test). Following euthanasia, the ratio of mass of the explanted gastrocnemius (GSC) and tibialis anterior (TA) muscles were compared to their contralateral controls to assess the impact of ACES on minimizing disuse atrophy.

Animals with stimulation facilitated by ACES demonstrated significant reductions in disuse atrophy as compared to their controls (p<0.05, Student's two-tailed heteroscedastic t-test). To quantify axonal regeneration, axon counts were performed in the proximal and distal segments of the sural, tibial, and peroneal nerves harvested distal to the site of repair. On the contralateral side, the proximal-distal axon count ratios were approximately one and not significantly different between control and experimental animals. However, on the affected side, the proximal-distal axon count was significantly higher in the experimental group in the sural and peroneal nerve (p<0.05, Student's two-tailed heteroscedastic t-test). Thus, advantageously, ACES can facilitate lead stabilization, improve axonogenesis, and support chronic PNS which, in turn, yields functional recovery and prevents disuse atrophy.

In one exemplary study, to characterize ACES-based hardware removal, the force required to extract the implanted electrodes was measured with and without triggered dissolution. In dissolved ACES cuffs, the peak force and tensile stress in the surrounding tissue were significantly lower (p=0.005 and p=0.0069 respectively, n=9/group) as compared to non-dissolved controls. In animals with dissolved injectable ACES, the force required for removal was significantly lower (p<0.04, two-tailed heteroscedastic t-test) at 1.66+/−0.9N while undissolved controls required 5.87+/−2.9N. In cases with undissolved scaffolds, significant stretching and tearing through tissue layers was observed as the electrodes were extracted. Dissection to the implant site revealed little to no trauma of the repaired nerve with dissolved ACES, while tearing of adhesions to the nerve and in two cases, a disrupted nerve repair was observed in the control.

Upon explant, adhesions to the explanted electrodes were quantified by a blinded plastic surgeon (0—no adhesion, 5—fully adhered to scar tissue). Those induced with the dissolution of ACES scored 0.2+/−0.44 while the controls scored 4.8+/−0.44 for both cuff and injectable embodiments of ACES. The significant diminution of adhesions on dissolved ACES leads suggests successful chronic inhabitance in the polymer mesh and effective ACES cavitation prior to removal. Histological analysis of cross and longitudinal sections of the nerve demonstrated no significant foreign body response and efficacious repair. In both injectable and cuff embodiments, cavities with clean margins where the electrodes resided can be seen, confirming the mechanism of function of the scaffold. Thus, ACES and its triggerable removal system confer the rehabilitative benefits of ES and facilitate a trauma-free removal.

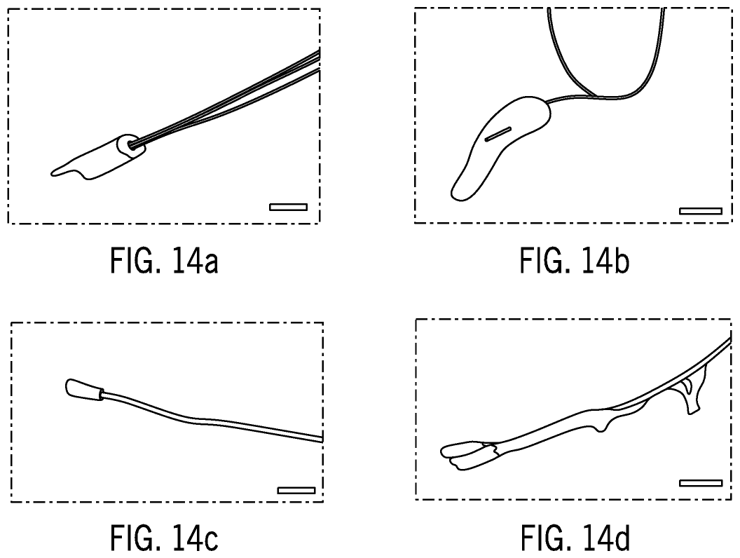

FIGS. 14a-d further illustrate exemplary results that show that removal of electrodes requires significantly less force when treated with gel dissolution versus those not treated. When not treated, removing electrodes can cause scaring. FIGS. 14a and 14c

FIGS. 14a and 14c are representative images of adhesions on electrodes removed from an experimental group treated with a gel dissolution and an ENC was dissolved prior to removal, and FIGS. 14b and 14d are representative images of adhesions on electrodes where an ENC was not dissolved prior to removal. Adhesions were found to a greater degree in the group represented in FIGS. 14b and 14d in which the scaffold was not dissolved prior to removal, suggesting greater trauma and disruption of tissues during the removal process.

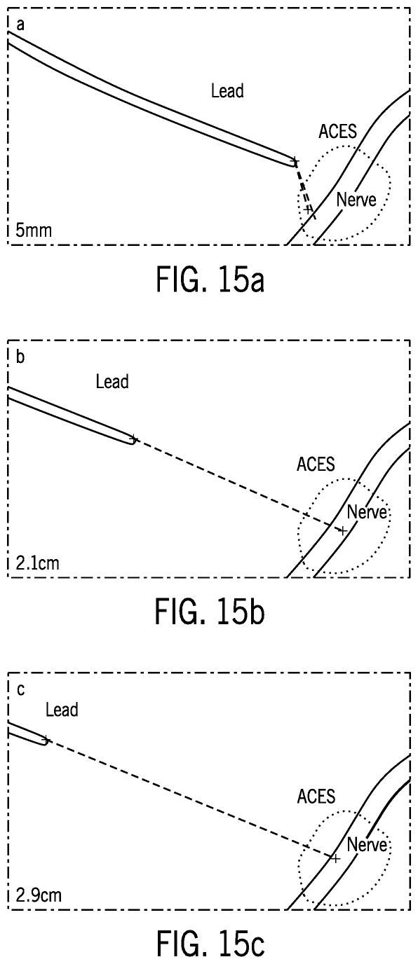

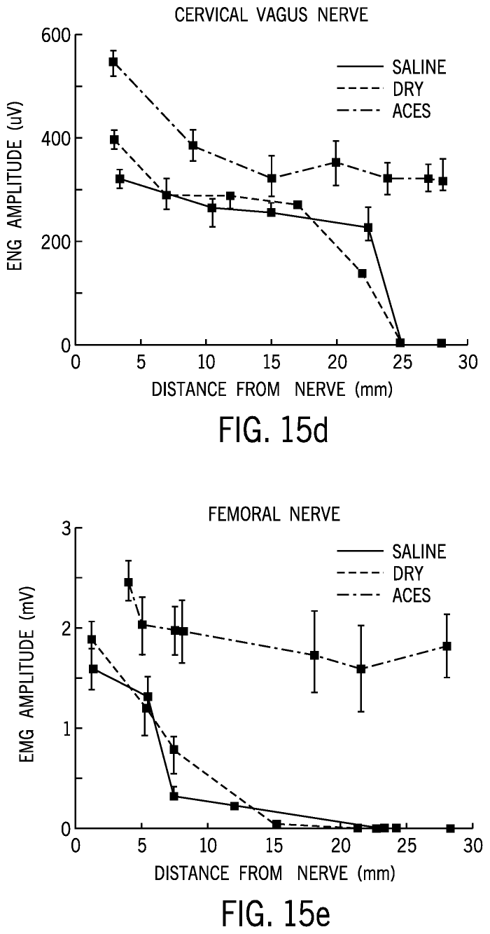

In another exemplary test, utility of ACES to stabilize leads and conduct stimulus for deeper anatomic targets was tested by percutaneously implanting electrodes near femoral and cervical vagus nerves. Under ultrasound guidance, a 14-gauge needle was advanced to the visualized hyperechoic femoral or vagus nerve in anesthetized swine (n=2). The electrode was placed through an introducer needle to the nerve followed by 1) no injection, 2) 3 mL saline injection or 3) 3 mL injectable ACES (FIG. 15a). The strength of contraction elicited in the distal muscle or nerve was measured in response to stimulation applied at distances of 2-28 mm from the nerve to assess stabilization and stimulation transmission (FIGS. 15a-c).

Under imaging, the gel and saline hydrodissected the neuromuscular fascial plane. However, saline dissipated quickly, contributing to a decline in stimulus transmission, whereas the gel maintained its shape for the duration of the experiment. Both gel and saline were hypoechoic, though depending on microbubble formation within the gel, varying degrees of echogenicity were visualized, aiding image-guided placement. In the dry condition, nearby off-target tissues were sometimes activated. However, in the presence of the ACES, stimulation evoked significantly higher EMG amplitudes in the target biceps femoris or distal vagus nerve (p<0.05, Student's two-tailed heteroscedastic t-test).

The ACES scaffold enabled PNS even when positioned more than 15 mm from the nerve, while the dry and saline conditions did not evoke a response (FIGS. 15d and 15e). Upon repeated actuation, the muscles demonstrated less than 10% reduction in force with over 25 pulses, suggesting no short-term neuromuscular fatigue or reduction in the gel's conductive capabilities. Upon euthanasia and dissection of the region, a semi-solid and connected path formed by ACES between the lead and the target nerve was observed, substantiating the existence of a conductive pathway enabling the observed stimulation-response results.

Embodiments of the disclosure provide a dissolvable electroceutical nerve scaffold (ENS) that can be pre-formed into a cuff (ENC) or gel (ENG) that is practical to insert at the site of nerve injury/disease and can be dissolved/removed atraumatically. ENS can enhance both motor and sensory function. This effective ENC device can translate electrical stimulation into clinical practice and improve the lives of patients and dependents agonized by loss of peripheral nerve function.

In some embodiments, an electrotherapeutic scaffold can be applied to a variety of clinical areas, including neuropathic pain management, peripheral nerve stimulation across nerve autograft/nerve allograft, organ stimulation for autonomic dysfunction such as bladder dysfunction, and stimulation of GI tract or pelvic floor.

Embodiments of the nerve repair system, and in particular, an absorbable conductive electrotherapeutic scaffold (ACES), can provide hassle-free, quick, and safe placement of leads as well as triggerable release with negligible tissue damage. By minimizing the number of adhesions to the electrode surfaces, the current amplitudes required to active neural tissues can also be minimized. By facilitating stable electrode placement and preventing lead migration, ACES makes electrical peripheral nerve stimulation (PNS) efficacious, which can provide improved sensory and motor function following nerve repair.

Conventional methods of nerve repair may include hollow nerve wraps; however, these conventional systems and methods provide no targeted acceleration of nerve regeneration or a mechanism to prevent the disuse atrophy Wallerian degradation that often outpace regeneration. Embodiments of the disclosure provide a nerve repair system that positions leads on both proximal and distal sides of a nerve repair site which can aid both directed axonogenesis and distal muscle stimulation.

Additionally, an absorbable conductive electrotherapeutic scaffold (ACES) according to embodiments of the disclosure can facilitate the neuromodulation of deep-set visceral autonomic nerve targets, which remain difficult to treat by conventional approaches. Historically, deep nerves have rarely been targeted given the degree of risk and morbidity associated with implantation and removal in open surgery. Instead, key nerves such as the celiac and superior mesenteric artery plexus, which critically influence gastric motility and pain, have been treated by either interventional ablation (celiac axis neurolysis) or endovascular ablation. With ACES, these nerves can be accessed via CT and/or ultrasound and the scaffolding will conform to the local anatomical features. Furthermore, given limitations in imaging resolution, potential gaps in lead placement, or lead migration within the scaffold are possible; these events can be compensated for by the conductive property of the scaffold. This enables minimally invasive image-guided placement, availing a wide range of applications.

Further, in some embodiments, hydrogels described herein could also be loaded with drugs, growth factors, and/or immune/neuronal modulators to add a pharmacologic dimension to the intervention. Beyond leads, ACES could be adapted to stabilize other hardware requiring temporary implantation and removal without tissue damage (e.g., catheters, pumps, expanders, depots), enabling new therapeutic interventions previously stifled by hardware implantation and removal challenges.

Within this specification embodiments have been described in a way which enables a clear and concise specification to be written, but it is intended and will be appreciated that embodiments may be variously combined or separated without parting from the invention. For example, it will be appreciated that all preferred features described herein are applicable to all aspects of the invention described herein.

Thus, while the invention has been described in connection with particular embodiments and examples, the invention is not necessarily so limited, and that numerous other embodiments, examples, uses, modifications and departures from the embodiments, examples and uses are intended to be encompassed by the claims attached hereto. The entire disclosure of each patent and publication cited herein is incorporated by reference, as if each such patent or publication were individually incorporated by reference herein.

Various features and advantages of the invention are set forth in the following claims.

1. A system for nerve repair comprising:
a polymer configured to form an elongated conduit forming a lumen configured to surround an in vivo nerve therein and provide a mechanical stabilization to the in vivo nerve; and
wherein the polymer is configured to degrade upon being subjected to a dissolution solution to remove the mechanical stabilization from the in vivo nerve.

2. The system of claim 1, wherein the polymer includes a hydrogel.

3. The system of claim 2, wherein the hydrogel includes alginate poly-acrylamide hydrogel.

4. The system of claim 1, wherein the polymer includes an interpenetrating network of (bis) acryloyl cystamine-crosslinked polyacrylamide and sodium alginate or calcium chloride.

5. The system of claim 1, wherein the polymer includes a mesh.

6. The system of claim 5, wherein the mesh is a knitted mesh.

7. The system of claim 1, further comprising a secondary gel layer disposed about at least a portion of a surface of the polymer defining the lumen.

8. The system of claim 7, wherein the secondary gel is loaded with at least one of a biologic, drug, or imaging marker configured to promote nerve growth, or counteract impediments to nerve growth, or aid visual identification of the in vivo nerve.

9. The system of claim 7, wherein the secondary gel includes a hyaluronanic acid or methylcellulose interpenetrating network.

10. The system of claim 9, wherein the interpenetrating network contains at least one of gold or Poly (lactic-co-glycolic acid) (PLGA) microparticles to at least one of increase conductivity or elute a therapeutic compound to promote nerve or tissue growth or to function as an immune or neuronal modulator.

11. The system of claim 1, further comprising at least two electrodes spaced apart along the lumen to deliver electrical stimulation to the in vivo nerve.

12. The system of claim 1, wherein the dissolution solution includes glutathione, sodium bicarbonate, or Ethylenediaminetetraacetic acid.

13. The system of claim 1, wherein the dissolution solution includes a 2:1 ratio of acrylamide to alginate.

14. The system of claim 1, wherein the polymer is formed as a shredded formulation that is configured to be injected percutaneously proximate to the in vivo nerve and surround the nerve to form the lumen.

15. A kit comprising:
a polymer configured to be formed into an elongated conduit surrounding an in vivo nerve in a lumen formed about the in vivo nerve to mechanically stabilize the in vivo nerve; and
a dissolution solution configured to dissolve the polymer and remove the mechanical stabilization from the in vivo nerve upon being delivered to the polymer.

16. The kit of claim 15, further comprising at least two electrodes configured to be spaced apart along the lumen to deliver electrical stimulation to the in vivo nerve.

17. The kit of claim 15, further comprising at least one of a biologic or other drug configured to promote nerve growth or counteract impediments to nerve growth.

18. The kit of claim 17, further comprising a secondary gel configured to engage the biologic or other drug and the polymer, at least along a portion of a surface forming the lumen.

19. A method of repairing a nerve injury, the method comprising:
arranging a polymer about an in vivo nerve to form an elongated conduit defining a lumen receiving an in vivo nerve therein and mechanically stabilize the in vivo nerve; and
delivering a dissolution solution to the polymer to remove the mechanical stabilization from the in vivo nerve.

20. The method of claim 19, further comprising positioning probes to deliver electrical or optical stimulation to the in vivo nerve before arranging the polymer to secure the probes to deliver the electrical or optical stimulation to the in vivo nerve when the polymer is mechanically stabilizing the in vivo nerve.

21. The method of claim 19, wherein arranging the polymer includes injecting the polymer as a gel to extend about the in vivo nerve.

22. A system for nerve repair comprising:
a stabilizing material configured to form an elongated conduit forming a lumen configured to surround an in vivo nerve therein and provide a mechanical stabilization to the in vivo nerve; and
wherein the stabilizing material is configured to degrade upon being subjected to a dissolution solution to remove the mechanical stabilization from the in vivo nerve without damaging the in vivo nerve.

23. The system of claim 22, wherein the stabling material includes at least one of a polymer, protein, polysaccharide, metal, lipid, ceramic, or other material that is configured to degrade upon being subjected to the dissolution solution without damaging the in vivo nerve.
